(19) United States
(12) Patent Application Publication
(10) Pub. No.: US 2016/0250040 A1
(43) Pub. Date: Sep. 1, 2016

(54) MEDICAL INSTRUMENTATION

(71) Applicant: Aesculap AG, Tuttlingen (DE)

(72) Inventors: Thomas Hermle, Rottweil (DE); Berna Richter, Muehlheim (DE)

(21) Appl. No.: 15/149,295

(22) Filed: May 9, 2016

Related U.S. Application Data
(63) Continuation of application No. PCT/EP2014/074252, filed on Nov. 11, 2014.

(30) Foreign Application Priority Data
Nov. 13, 2013 (DE) 10 2013 112 496

(51) Int. Cl. A61F 2/46; A61B 34/10; A61B 17/17

(52) U.S. Cl. CPC A61F 2/4609; A61F 2/4657; A61B 17/1746; A61B 34/10; A61F 2002/4687; A61F 2002/4668; A61B 2034/108; A61B 2017/568

(57) ABSTRACT

The present invention relates to a medical instrumentation, in particular, for implanting an acetabular cup, comprising a medical alignment instrument with a shaped body, which has a patient-specific bone abutment surface facing away from the shaped body and deviating from a spherical surface section, the bone abutment surface being shaped so as to correspond to a bone surface of the patient, wherein the shaped body has at least one viewing edge with a viewing height, and wherein the viewing edge adjoins the patient-specific bone abutment surface, and wherein a transition between the viewing edge and the patient-specific bone abutment surface is tangentially discontinuous.

### CROSS-REFERENCE TO RELATED APPLICATIONS

This application is a continuation of international application number PCT/EP2014/074252 filed on Nov. 11, 2014 and claims the benefit of German application number 10 2013 112 496.8 filed on Nov. 13, 2013, which are incorporated herein by reference in their entirety and for all purposes.

### FIELD OF THE INVENTION

The present invention relates to medical instrumentation, in particular, for implanting an acetabular cup generally, and more specifically to a medical instrumentation, in particular, for implanting an acetabular cup, comprising a medical alignment instrument with a shaped body, which has a patient-specific bone abutment surface facing away from the shaped body and deviating from a spherical surface section, the bone abutment surface being shaped so as to correspond to a bone surface of the patient.

### BACKGROUND OF THE INVENTION

In particular, the optimal positioning of the acetabular cup of the hip joint endoprosthesis to be implanted is important for treatment of a patient with a damaged hip to be successful. For this purpose, it is, for example, known to palpate landmarks on the pelvic bone and thereby determine a reference plane of the pelvis with respect to which all parts of the hip joint endoprosthesis are aligned and implanted.

In particular, in obese patients, the palpation of landmarks is error-prone as the bone cannot always be precisely palpated underneath the tissue. Another problem in the palpation of landmarks is that they must be easy to reach. This is not the case, in particular, during surgery in lateral position.

Furthermore, it is known to use referencing devices in connection with navigation systems, in particular, in order to implant the acetabular cup and the hip shaft in a defined manner. Use of these is, however, quite time-consuming.

### SUMMARY OF THE INVENTION

In a first aspect of the invention, a medical instrumentation, in particular, for implanting an acetabular cup comprises a medical alignment instrument with a shaped body, which has a patient-specific bone abutment surface facing away from the shaped body and deviating from a spherical surface section. The bone abutment surface is shaped so as to correspond to a bone surface of the patient. The shaped body has at least one viewing edge with a viewing height, and the viewing edge adjoins the patient-specific bone abutment surface, and a transition between the viewing edge and the patient-specific bone abutment surface is tangentially discontinuous.

### BRIEF DESCRIPTION OF THE DRAWINGS

The foregoing summary and the following description may be better understood in conjunction with the drawing figures. There are shown in:

FIG. 1 a schematic perspective partial view of the pelvis with alignment instrument positioned on the acetabulum;

FIG. 2 an exploded representation of the arrangement from FIG. 1;

FIG. 3 a sectional view taken along line 3-3 in FIG. 1;

FIG. 4 a perspective view of the shaped body of the alignment instrument from the proximal direction;

FIG. 5 a perspective view of the shaped body from FIG. 4 from the distal direction;

FIG. 6 a schematic perspective view similar to FIG. 1 with a second embodiment of a shaped body;

FIG. 7 a schematic exploded representation of the arrangement from FIG. 6;

FIG. 8 a sectional view taken along line 8-8 in FIG. 6;

FIG. 9 a schematic perspective view of the shaped body from FIG. 7 from the proximal direction;

FIG. 10 a schematic perspective view of the shaped body from FIG. 9 from the distal direction;

FIG. 11 a schematic perspective view similar to FIG. 1 with a further embodiment of a shaped body;

FIG. 12 a schematic exploded representation of the arrangement from FIG. 11;

FIG. 13 a sectional view taken along line 13-13 in FIG. 11;

FIG. 14 a schematic perspective view of the shaped body from FIG. 12 from the proximal direction;

FIG. 15 a schematic perspective view of the shaped body from FIG. 14 from the distal direction;

FIG. 16 a partially broken-open view of the grip element from FIGS. 2, 7 and 12.

### DETAILED DESCRIPTION

Although the invention is illustrated and described herein with reference to specific embodiments, the invention is not intended to be limited to the details shown. Rather, various modifications may be made in the details within the scope and range of equivalents of the claims and without departing from the invention.

The present invention relates to a medical instrumentation, in particular, for implanting an acetabular cup, comprising a medical alignment instrument with a shaped body, which has a patient-specific bone abutment surface facing away from the shaped body and deviating from a spherical surface section, the bone abutment surface being shaped so as to correspond to a bone surface of the patient, wherein the shaped body has at least one viewing edge with a viewing height, and wherein the viewing edge adjoins the patient-specific bone abutment surface, and wherein a transition between the viewing edge and the patient-specific bone abutment surface is tangentially discontinuous.

The solution proposed in accordance with the invention prevents the alignment instrument with the bone abutment surface from not being fully positioned on the bone surface, for example, an area of the acetabulum of the patient. In particular, if the bone abutment surface is of such size that the entire acetabulum is used as reference, the surgeon no longer has any view into the acetabulum and, therefore, no longer any orientation. He then has to rely exclusively on manually feeling whether the shaped body is seated correctly, i.e., with surface-to-surface contact, in the acetabulum. It is particularly difficult to tactilely sense this if a bearing surface is relatively large. Also, a large bearing surface will increase the risk of inaccuracies on the patient-specific bone abutment surface or foreign bodies at the operating site preventing a definite and precise seating of the shaped body on the acetabulum. An incorrectly positioned shaped body does, however, mean that the surgeon will use a wrong reference for the implantation, which may result in seriously incorrect positioning of the implant. Precisely these problems are prevented by the solution proposed in accordance with the invention. The viewing edge constructed in the indicated manner enables the surgeon to maintain at least a limited view into the acetabulum and, therefore, not only manually but also optically check the reference during use of the alignment instrument. Owing to the viewing edge adjoining the patient-specific bone abutment surface, the surgeon is able not only to feel but also to see the optimal positioning of the shaped body on the acetabulum. In particular, owing to the viewing edge adjoining the patient-specific bone abutment surface, the surgeon is able to clearly recognize and, in particular, also check with palpation instruments, for example, a palpation hook, whether the shaped body with the patient-specific bone abutment surface is seated in the predefined manner in the acetabulum. If this is the case, the alignment instrument can then be used, in particular, by setting reference pins or by referencing by means of a navigation system, to clearly determine the position of the patient's pelvis. Owing to the precision of the bone abutment surface, which is preferably manufactured on the basis of noninvasively determined bone contour data of the patient, a simple mechanical alignment aid is thus obtained for determining and referencing the position not only of the pelvis, but, in particular, of the acetabulum. The shaped body may have, in particular, two, three, four or even more viewing edges. These may extend at least partially parallel or at a defined angle relative to one another. In particular, ring-shaped viewing edges closed within themselves, for example, in the form of inner boundary surfaces of through-openings of the shaped body are also possible.

A particularly safe checking of the exact position of the shaped body in the acetabulum is made possible, in particular, by the at least one viewing edge having a width or height which corresponds at least to a minimum thickness of the shaped body.

For the stability and, in particular, palpability of the viewing edge it is expedient for the at least one viewing edge to have a width or height which corresponds at most to three times a minimum thickness of the shaped body.

In accordance with a further preferred embodiment of the invention, it may be provided that the at least one viewing edge forms a flat or substantially flat surface area or forms a boundary surface of a recess of the shaped body, which recess at least partially penetrates the bone abutment surface. For example, the bone abutment surface can face away from the shaped body and face in the distal direction. In particular, the recess can then be formed on the side of the shaped body and extend as far as the patient-specific bone abutment surface.

Furthermore, the shaped body may have an edge and optionally also a surface area which is not patient-specifically shaped. For example, this surface area may define a section of a surface of an ellipsoid, in particular, a sphere. This surface area may, for example, be formed on a carrier which forms part of the shaped body on which the patient-specific bone abutment surface is formed on a projection facing in the distal direction.

It is expedient for the at least one recess to be constructed in the form of a through-opening and for the at least one viewing edge to form an inner surface of the through-opening. In particular, one, two, three, four or more through-openings may be provided on the shaped body. These enable the surgeon to see the acetabulum through the shaped body and, in particular, owing to the direct view of the viewing edges bordering the through-openings to check optically whether or not the shaped body is positioned in the desired manner on the acetabulum.

It is advantageous for the alignment instrument to define an instrument longitudinal axis and for a longitudinal axis of the through-opening to run parallel or substantially parallel to the instrument longitudinal axis. The surgeon is thus provided with optimum optical access to the acetabulum through the shaped body.

The through-opening can be manufactured particularly easily if it is constructed in the form of a borehole.

To be able to check the position of the shaped body on the acetabulum as best as possible independently of an access and a concrete operative situation, it is advantageous for the alignment instrument to have two, three, four or more recesses. In particular, any combinations of flat or substantially flat viewing edges with recesses, in particular, in the form of through-openings, are conceivable.

Furthermore, it is advantageous for the bone abutment surface to be at least partially adapted to a contour of the acetabulum of the patient. The alignment instrument can thus be precisely positioned on the acetabulum as the latter normally has a shape which deviates from a rotational symmetry and allows the positioning of a patient-specific bone abutment surface adapted to the bone contour only in precisely one defined position.

The bone abutment surface preferably has a contour which corresponds at least to a portion of the fossa and/or to a portion of the acetabular notch. In particular, the two aforementioned structures, i.e., the fossa and the acetabular notch, provide in a patient-specific manner an optimum reference for the alignment of the acetabular cup to be implanted on the pelvic bone of the patient. With this simple mechanical, precise alignment aid, alignment or attachment pins can then be optionally positioned and fixed to the pelvic bone or the position of the alignment instrument, in particular, of the shaped body, can also be referenced by means of a navigation system, for example, using a navigated palpation instrument.

It is expedient for at least one attachment element receptacle to be arranged or formed on the shaped body. In particular, two, three or more attachment element receptacles may be provided. In particular, these are constructed so as to correspond to attachment pins, which may optionally serve to fix the shaped body to the pelvic bone or to also fix a referencing device with corresponding marker elements for referencing the pelvis by means of a surgical navigation system.

It is expedient for the shaped body to be of dish-shaped construction and to have a constant or substantially constant thickness. It can, therefore, be manufactured particularly easily, for example, by molding or 3-D printing. In addition, it is, therefore, light and takes up only a small volume in the area of the operating site. Furthermore, a width or height of the viewing edge is not too large, which, in particular, might impair a view of the transition between the viewing edge and the patient-specific bone abutment surface.

It is advantageous for the bone abutment surface and/or the shaped body to be manufactured by molding or 3-D printing. The shaped body can thus be manufactured in an easy and cost-effective way, in particular, from a plastic material, preferably a sterilizable plastic material. Optionally, the shaped body or the bone abutment surface may also be made of a metal.

In accordance with a further preferred embodiment of the invention, it may be provided that the bone abutment surface of the shaped body defines bone abutment surface contour data which correspond or substantially correspond to noninvasively determined bone contour data of the patient. In particular, the bone contour data of the patient may be data from X-ray or magnetic resonance images. These can be determined, in particular, by corresponding X-ray and magnetic resonance devices in digital form and then directly further processed to form printing data or for a CAD-controlled reamer, for preparing a mold or for directly printing the bone abutment surface or the shaped body with a 3-D printer.

The alignment instrument is particularly easy to handle if it has a grip element.

The grip element is expediently constructed so as to be releasably connectable to the shaped body. The shaped body can, for example, thus be positioned with the grip element on the pelvic bone and temporarily fixed there with attachment elements, for example, bone pins or bone screws.

To improve the view of the operating site, the grip element can then be optionally released from the shaped body.

The grip element and the shaped body can be connected to each other and also released from each other in a particularly easy way if the alignment instrument has a coupling device for temporarily coupling the shaped body to the grip element.

The grip element and the molded body can be connected to each other in a particularly easy and secure way if the coupling device comprises at least one first coupling element and at least one second coupling element, and if the at least one first and second coupling elements are constructed so that they correspond to each other and are in engagement in a coupling position and in disengagement in a release position.

The connecting of the grip element to the shaped body is particularly easy, in particular, also when there is no direct view of the operating site, if the at least one first coupling element is constructed in the form of a coupling receptacle and if the at least one second coupling element is constructed in the form of a coupling projection. The coupling receptacle may be selectively constructed on the grip element or on the shaped body.

To achieve as high a stability as possible with the connection of the two coupling elements in a coupling position, it is advantageous for the at least one first coupling element to have an internal thread section and for the at least one second coupling element to have a corresponding external thread section. In particular, this configuration makes it possible for the grip element to be screwed onto the shaped body or screwed off it.

One of the two coupling elements is preferably arranged or constructed on the shaped body and the other one of the two coupling elements on the grip element. In particular, the at least one first coupling element can be arranged or constructed on the shaped body, and the at least one second coupling element on the grip element.

The handling of the alignment instrument can be further improved by the grip element having an elongate shaft and by a gripping area being arranged or formed at a proximal end area of the shaft. A surgeon can thus easily and safely hold the grip element at the gripping area. The gripping area may, for example, be ergonomically designed.

Furthermore, it is advantageous for the shaft to define a longitudinal axis, and for a proximal end face of the shaft to be constructed in the form of an impact surface for an impactor tool. It is thus possible to use the grip element, in particular, to drive, for example, the shaped body into the acetabulum.

The impact surface is expediently at least partially flat and defines a plane extending perpendicularly to the longitudinal axis. An impact pulse can thus be easily and safely exerted with an impactor tool on the grip element, more particularly, with a minimal risk of the impactor tool sliding off the impact surface.

Furthermore, it is advantageous for the shaft to be of at least two-part construction and to comprise a shaft sleeve and an inner shaft guided in the shaft sleeve, and for the second coupling element to be arranged or formed at a distal end of the inner shaft or at a distal end of the shaft sleeve. In particular, the inner shaft can be rotatably and/or displaceably mounted in the shaft sleeve and coupled to an actuating element, for example, a rotary knob. This makes it possible, for example, for the surgeon to hold the grip element at the gripping area which, in particular, can be rotationally fixedly connected to the shaft sleeve or constructed as part thereof, and to then rotate the inner shaft relative to the shaft sleeve. For example, the inner shaft can thus, if its distal end is provided with an external thread section, be screwed into a corresponding coupling receptacle with internal thread on the shaped body. This merely requires rotation of, for example, the inner shaft, but not of the grip element or of the shaft sleeve as a whole.

In order to press the shaped body with a defined force into the acetabulum, it is expedient for the instrumentation to also comprise an impactor tool.

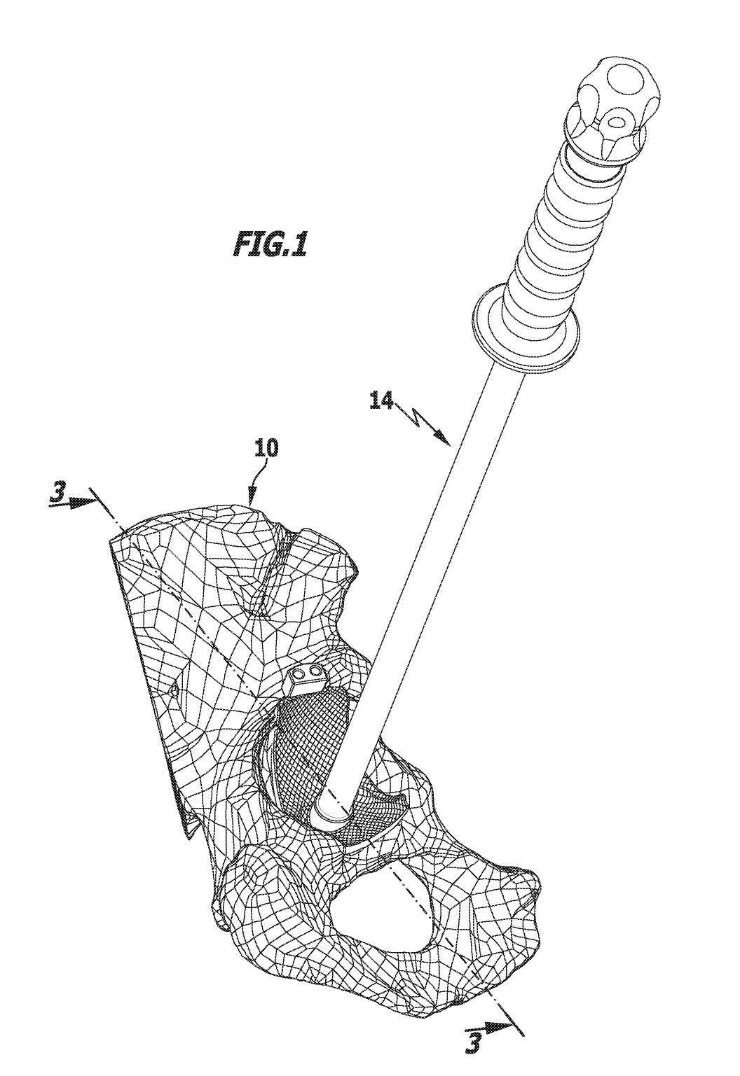

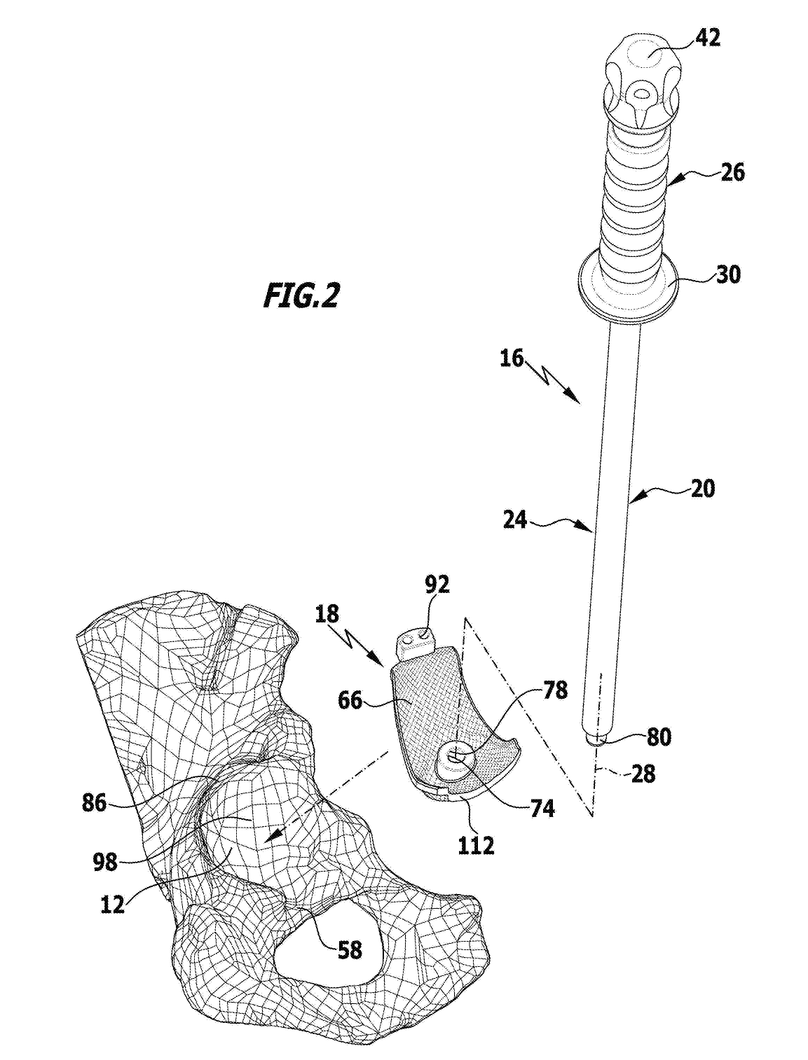

A section of a human pelvic bone 10 in which a hip joint endoprosthesis is to be implanted is shown schematically in FIG. 1. In particular, an acetabulum 12, which is also referred to as hip joint socket or pelvic socket and forms the bony portion of the natural hip joint formed in the anatomy by the pelvic bone 10, is shown in FIG. 2.

A medical instrumentation designated in its entirety by reference numeral 14 serves for positionally accurate alignment of an artificial acetabular cup, not shown in the Figures, of a hip joint endoprosthesis. The medical instrumentation comprises, in particular, a medical alignment instrument 16 with a shaped body 18 and a grip element 20.

The grip element 20 can be used, in particular, as impactor 22. It comprises an elongate shaft 24, at the proximal end of which a gripping area 26 is arranged or formed. A ring flange 30 protruding in the radial direction in relation to a longitudinal axis 28 delimits the gripping area 26 at the distal side and serves, in particular, to prevent a hand of the surgeon holding the grip element 20 from sliding down in the distal direction.

The shaft 24 comprises an elongate shaft sleeve 32 which is provided with a longitudinal borehole extending coaxially with the longitudinal axis 28. An elongate inner shaft 34 is rotatably and/or displaceably mounted in the longitudinal borehole. A proximal end of the inner shaft 34 is rotationally fixedly coupled to an actuating member 36 in the form of a rotary knob 38.

An end face 40 of the rotary knob 38 facing in the proximal direction forms an at least partially flat impact surface 42, which defines a plane 44 extending perpendicularly to the longitudinal axis 28. A distal end 46 of the inner shaft 34 projects over a distal end 48 of the shaft sleeve 32 and faces in the distal direction. Starting from the end 46, a short external thread section 50 is formed on the inner shaft 34. As a result of rotation of the rotary knob 38, the inner shaft 34 is also rotated relative to the shaft sleeve 32 about the longitudinal axis 28.

The shaped body 18 is of substantially dish-shaped construction and has an almost constant wall thickness. On its underside 52 facing in the distal direction, the shaped body 18 has a patient-specific bone abutment surface 54. This corresponds to the acetabulum 12 or at least to the fossa 56 specific for each patient and/or to the acetabular notch 58. The bone abutment surface 54 deviates from a spherical surface section.

The bone abutment surface 54 on the shaped body 18 and/or the shaped body 18 are preferably manufactured by molding or 3-D printing. For the molding, a mold is formed, for example, on the basis of noninvasively determined bone contour data of the patient. For example, the bone contour data may originate from X-ray and/or magnetic resonance images of the acetabulum 42 of the patient. Directly digitalized X-ray images can, for example, be further processed to form printing data for a 3-D printer, with which the bone abutment surface 54 can then be printed on a carrier or as part of the shaped body 18 together with the latter. In this way, a bone abutment surface 54 can then be constructed, which defines the bone abutment surface contour data corresponding to the noninvasively determined bone contour data of the patient.

The shaped body 18 also has a plurality of viewing edges 60, 62 and 64 in the form of recesses 112 or flattened regions as surface areas with a viewing height, which adjoin the patient-specific bone abutment surface 54. A transition between the viewing edges 60, 62 and 64 and the bone abutment surface 54 is tangentially discontinuous. The viewing edges 60, 62 and 64 are each of flat or substantially flat construction. The viewing edges 60 and 64 extend almost parallel to each other.

A peg 68 facing in the proximal direction is formed on an upper side 66 of the shaped body facing in the proximal direction. The peg 68 has a blind hole 70 which is provided with an internal thread section 72. The internal thread section 72 is formed so as to correspond to the external thread section 50.

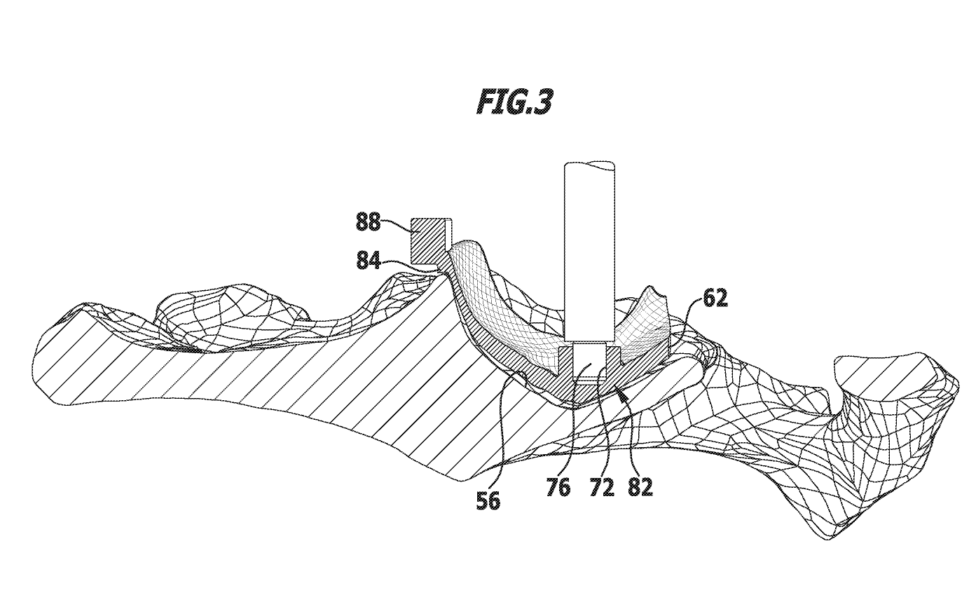

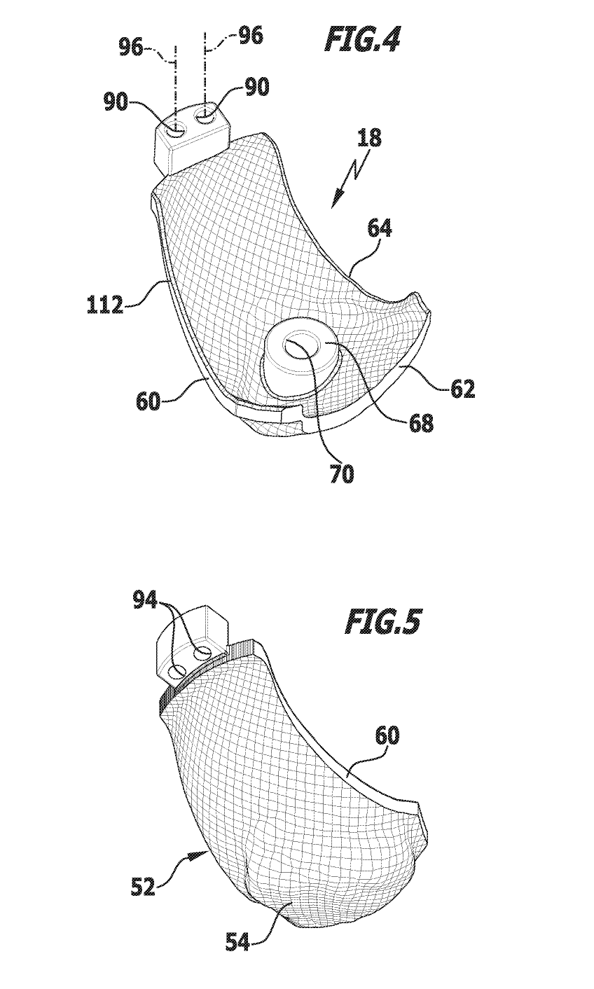

The blind hole 70 forms a coupling receptacle 74 which is constructed so as to correspond to a coupling projection 76 formed by the end 46. The coupling receptacle 74 and the coupling projection 76 form first and second coupling elements 78 and 80 of a coupling device designated in its entirety by reference numeral 82 for temporarily coupling the shaped body 18 to the grip element 20 in a coupling position as shown schematically, for example, in FIGS. 1 and 3.

There is also formed on the shaped body 18 an edge section 84 which engages over an edge 86 of the acetabulum 12 opposite the acetabular notch.

An attachment body 88, which is of substantially parallelepipedal construction, can be optionally arranged or formed on the section 84. Two attachment element receptacles 90 in the form of through-openings 92, which are manufactured as boreholes 94, are formed on the attachment body 88. Attachment element receptacle longitudinal axes 96 preferably run parallel to the longitudinal axis 28.

In particular, attachment pins can be introduced through the attachment element receptacles 90 into the pelvic bone 10, in order to, on the one hand, fix the alignment instrument 16 on the acetabulum 12 and/or to fix referencing devices with marker elements on them for determining the position of the shaped body 18 on the pelvic bone 10 and, therefore, its position in space by means of a navigation system.

As can be clearly seen, in particular, in FIGS. 1 and 3, the provision of the viewing edges 60, 62 and 64 enables a surgeon to check by sight or by means of a palpation hook the positionally correct arrangement of the shaped body 18 in the acetabulum 12. Only when the shaped body 18 with the bone abutment surface 54 lies with surface-to-surface contact against the bone surface 98 on the pelvic bone 10, do the position and alignment of the shaped body 18 correspond to the, in particular, noninvasively determined bone contour data.

With the aid of the alignment instrument 16, the acetabulum 12 can now be prepared as cup bearing for the acetabular cup of the hip joint endoprosthesis to be implanted using further instruments and tools.

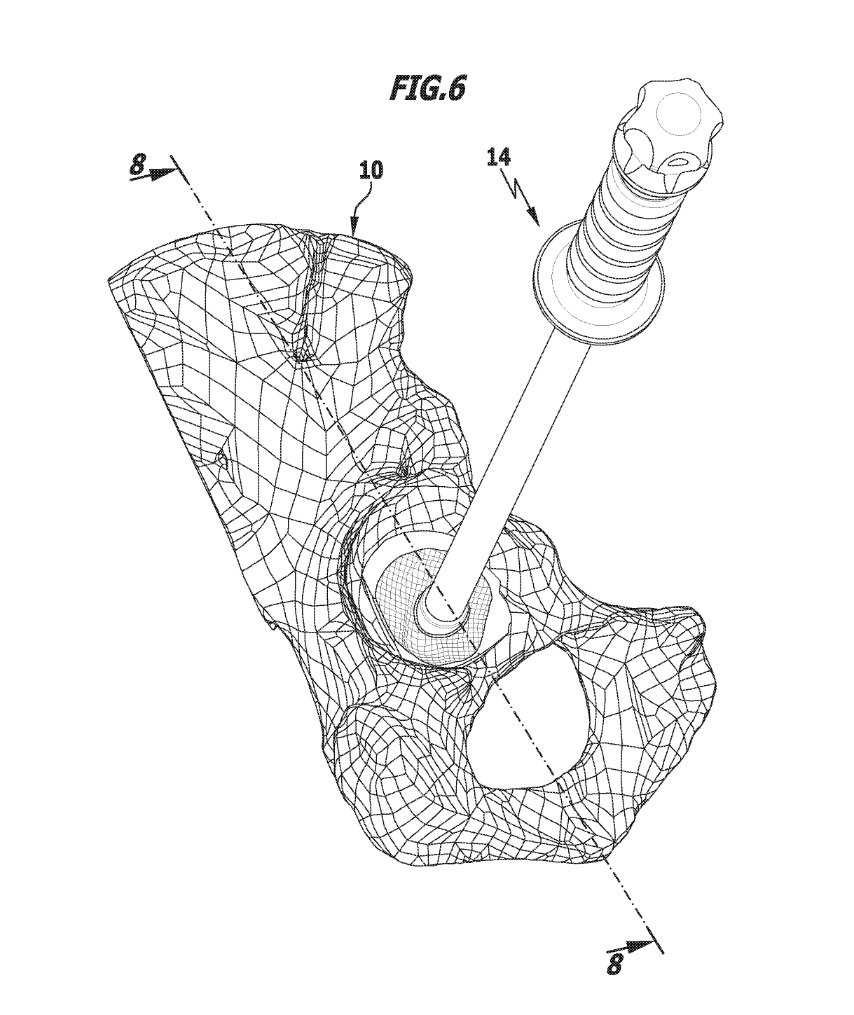

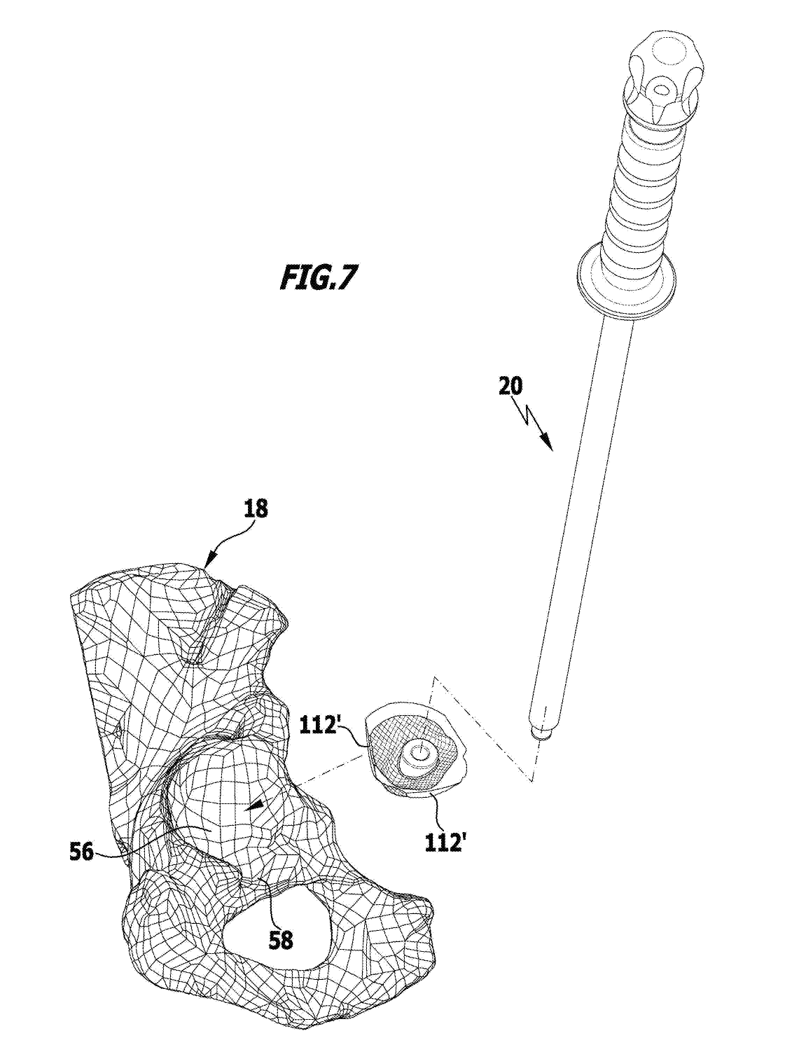

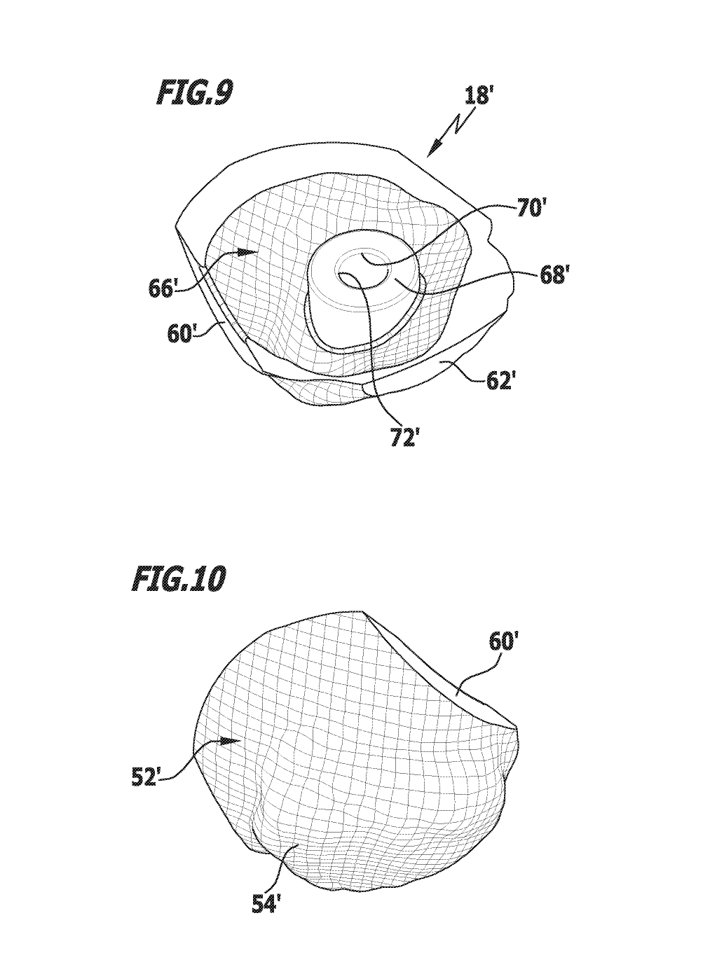

A second embodiment of a shaped body designated by reference numeral 18′ is shown in FIGS. 6 to 10. It forms part of the instrumentation 14. For the sake of clarity, the same parts and elements of the shaped body 18′ are given corresponding reference numerals which are used in the shaped body 18.

The shaped body 18′ is also of dish-shaped construction and has on its underside 52′ a bone abutment surface 54′, which corresponds to a negative image of the fossa 56. Also provided on the shaped body 18′ are recesses 112′ or flattened regions in the form of flat viewing edges 60′ and 62′, which are orientated substantially perpendicularly to each other and define planes extending substantially parallel to the longitudinal axis 28.

Also protruding on an upper side 66′ of the shaped body 18′ is a peg 68′, which is provided with a blind hole 70′ which has an internal thread section 72′. The shaped body 18′ can thus be coupled in a manner in analogy with the shaped body 18 to the grip element 20 as described above.

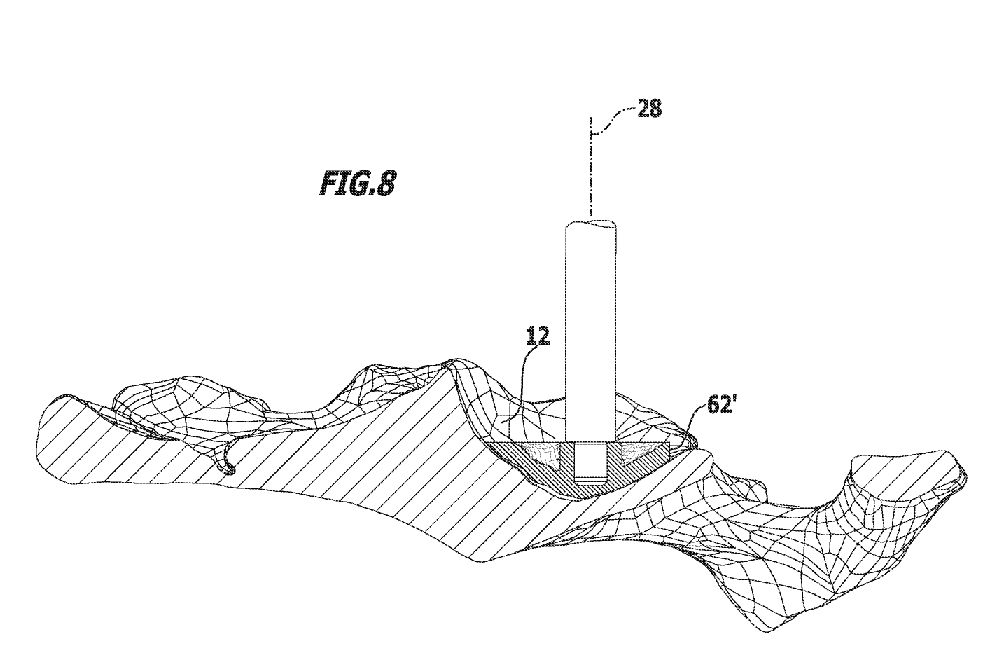

As can be clearly seen, in particular, in FIGS. 6 and 8, the viewing edges 60′ and 62′ enable a surgeon to optically check or to check together with a palpation instrument, for example, a palpation hook, whether the shaped body 18′ is positioned with a precise fit in the acetabulum 12.

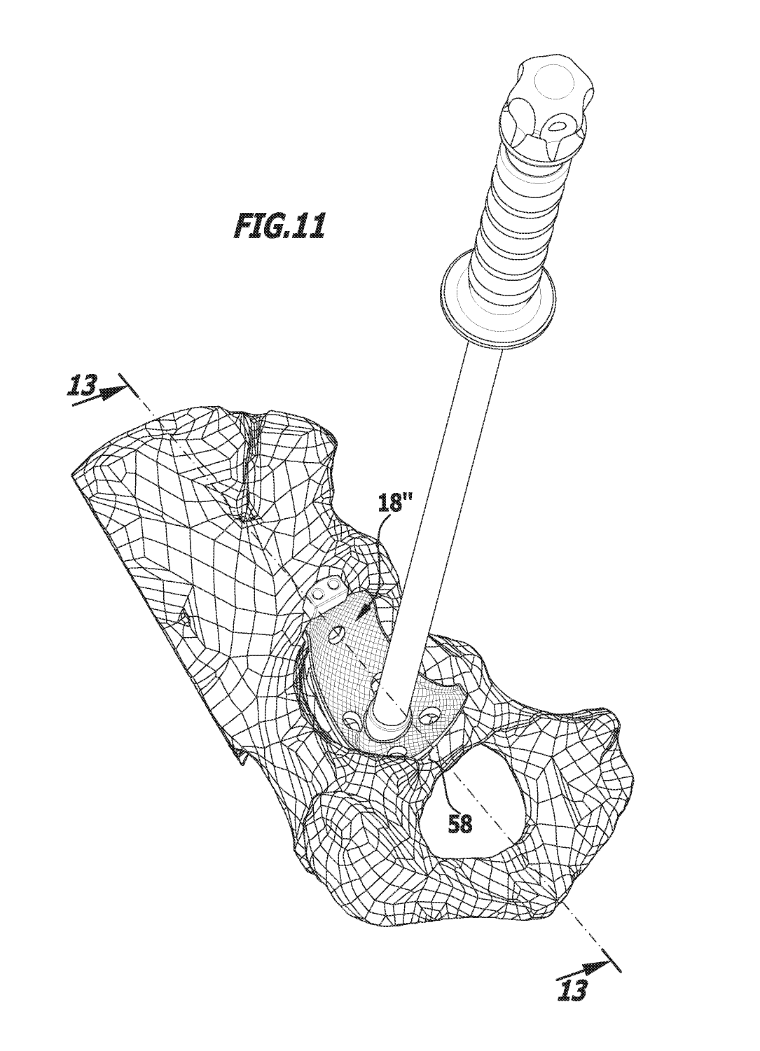

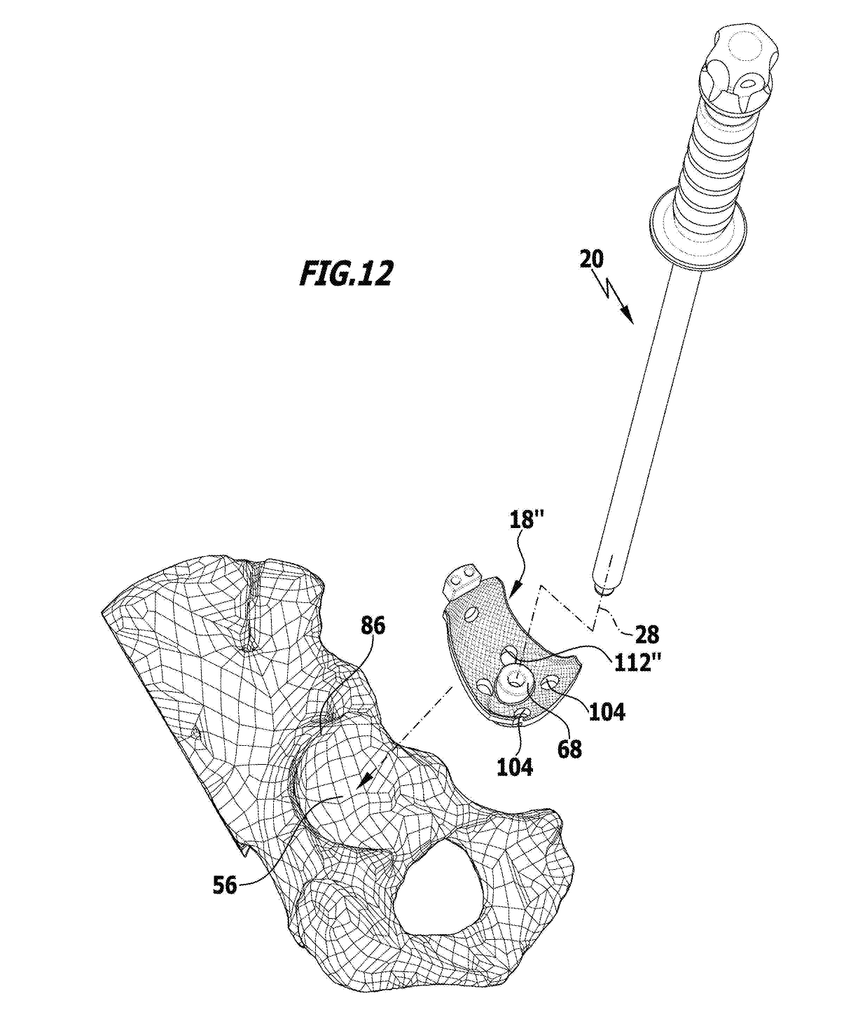

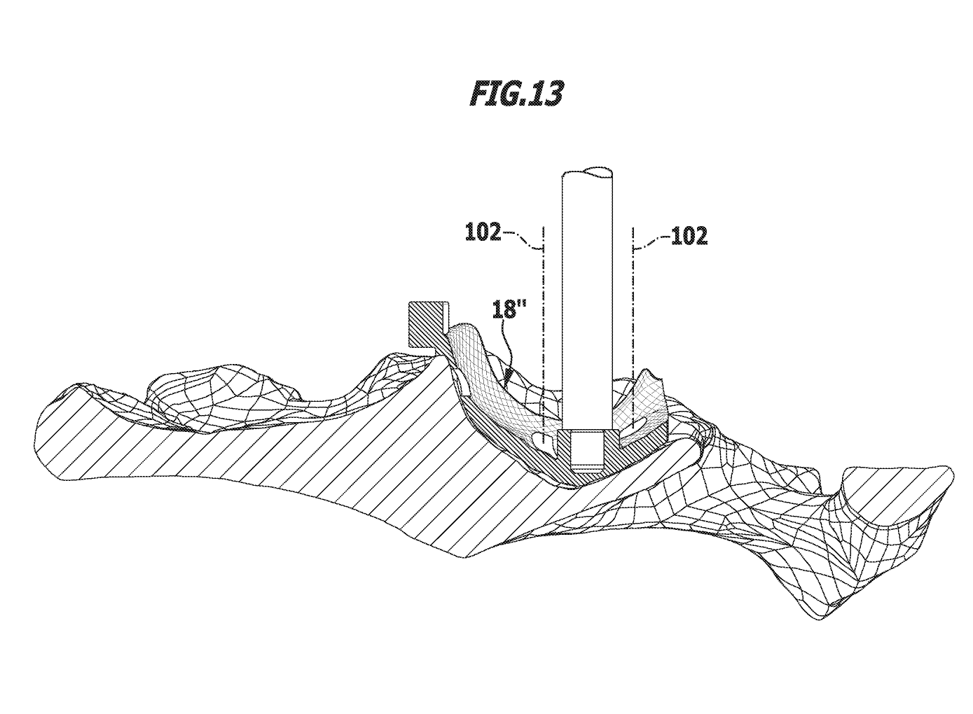

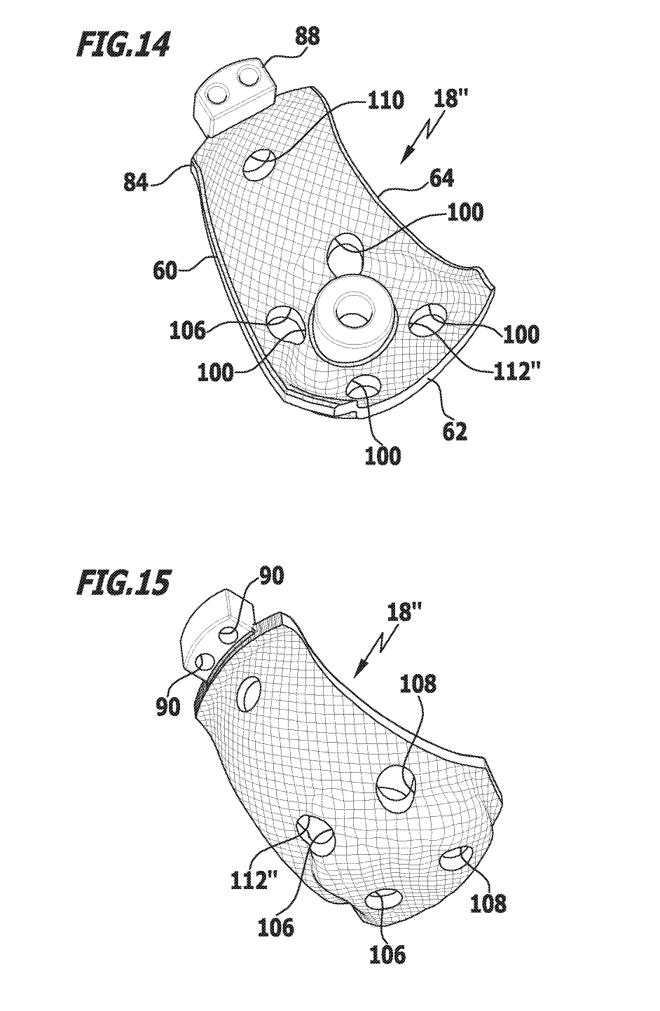

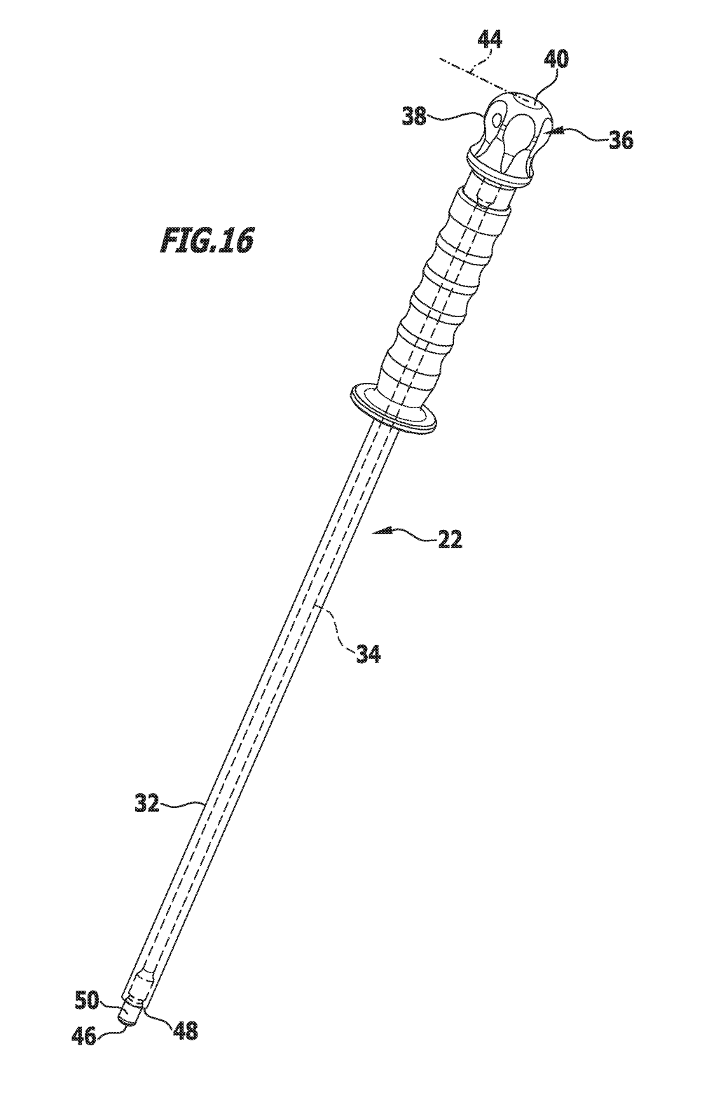

A third embodiment of a shaped body, designated in its entirety by reference numeral 18″, of the instrumentation 14 is shown schematically in FIGS. 11 to 15. The construction of the shaped body 18″ corresponds substantially to the construction of the shaped body 18, and so the same reference numerals were used for the shaped body 18″ as for the shaped body 18.

The shaped body 18″ differs from the shaped body 18′ in that it has a total of four recesses 112″ in the form of through-openings 100 arranged substantially uniformly around the peg 68. The through-openings 100 define through-opening longitudinal axes 102, which run substantially parallel to the longitudinal axis 28. The through-openings 100 are constructed in the form of boreholes 104. Inner surfaces 106 of the boreholes 104 form viewing edges 108, which enable a direct view of the acetabulum 12. The through-openings 100 can thus be used, in particular, as viewing window by a surgeon, in order to check by sight the optimal positioning of the shaped body 18″ on the acetabulum 12.

Optionally, further through-openings 110 may also be provided on the shaped body 18″. These may be constructed in the form of boreholes outside of the patient-specific bone abutment surface 54.

The shaped bodies 18, 18′ and 18″ can all be manufactured from a plastic material or a metal by molding or 3-D printing.

### LIST OF REFERENCE NUMERALS

10: pelvic bone
12: acetabulum
14: instrumentation
16: alignment instrument
18: shaped body
20: grip element
22: impactor
24: shaft
26: gripping area
28: longitudinal axis
30: ring flange
32: shaft sleeve
34: inner shaft
36: actuating member
38: rotary knob
40: end face
42: impact surface
44: plane
46: end
48: end
50: external thread section
52: underside
54: bone abutment surface
56: fossa
58: acetabular notch
60: viewing edge
62: viewing edge
64: viewing edge
68: peg
70: blind hole
72: internal thread section
74: coupling receptacle
76: coupling projection
78: first coupling element
80: second coupling element
82: coupling device
84: edge section
86: edge
88: attachment body
90: attachment element receptacle
92: through-openings
94: borehole
96: attachment element receptacle
98: bone flange
100: through-opening
102: through-opening longitudinal axis
104: borehole
106: inner surface
108: viewing edge
110: through-opening
112: recess

What is claimed is:

1. Medical instrumentation, in particular, for implanting an acetabular cup, comprising a medical alignment instrument with a shaped body, which has a patient-specific bone abutment surface facing away from the shaped body and deviating from a spherical surface section, the bone abutment surface being shaped so as to correspond to a bone surface of the patient, wherein the shaped body has at least one viewing edge with a viewing height, and wherein the viewing edge adjoins the patient-specific bone abutment surface, and wherein a transition between the viewing edge and the patient-specific bone abutment surface is tangentially discontinuous.

2. Medical instrumentation in accordance with claim 1, wherein the at least one viewing edge has a width or height which corresponds at least to a minimum thickness of the shaped body.

3. Medical instrumentation in accordance with claim 2, wherein the at least one viewing edge has a width or height which corresponds at most to three times a minimum thickness of the shaped body.

4. Medical instrumentation in accordance with claim 1, wherein the at least one viewing edge forms a flat or substantially flat surface area or forms a boundary surface of a recess of the shaped body, which recess at least partially penetrates the bone abutment surface.

5. Medical instrumentation in accordance with claim 4, wherein the at least one recess is constructed in the form of a through-opening, and wherein the at least one viewing edge forms an inner surface of the through-opening.

6. Medical instrumentation in accordance with claim 5, wherein at least one of
the alignment instrument defines an instrument longitudinal axis, and a longitudinal axis of the through-opening runs parallel or substantially parallel to the instrument longitudinal axis
and
the through-opening is constructed in the form of a borehole.

7. Medical instrumentation in accordance with claim 4, comprising two, three, four or more recesses.

8. Medical instrumentation in accordance with a claim 1, wherein the bone abutment surface at least one of
is at least partially adapted to a contour of the acetabulum of the patient and
has a contour which corresponds at least to a portion of the fossa and/or to a portion of the acetabular notch.

9. Medical instrumentation in accordance with claim 1, wherein at least one of
at least one attachment element receptacle is arranged or formed on the shaped body
and
the shaped body is of dish-shaped construction and has a constant or substantially constant thickness.

10. Medical instrumentation in accordance with claim 1, wherein at least one of
at least one of the bone abutment surface and the shaped body are manufactured by molding or 3-D printing
and
the bone abutment surface of the shaped body defines bone abutment surface contour data which correspond or substantially correspond to noninvasively determined bone contour data of the patient, in particular, bone contour data of the patient from X-ray or magnetic resonance images.

11. Medical instrumentation in accordance with claim 1, characterized by a grip element.

12. Medical instrumentation in accordance with claim 11, wherein the grip element is constructed so as to be releasably connectable to the shaped body.

13. Medical instrumentation in accordance with claim 1, characterized by a coupling device for temporarily coupling the shaped body to the grip element.

14. Medical instrumentation in accordance with claim 13, wherein the coupling device comprises at least one first coupling element and at least one second coupling element, and wherein the at least one first and second coupling elements are constructed so that they correspond to each other and are in engagement in a coupling position and in disengagement in a release position.

15. Medical instrumentation in accordance with claim 14, wherein the at least one first coupling element at least one of
is constructed in the form of a coupling receptacle and the at least one second coupling element in the form of a coupling projection
and
has an internal thread section and the at least one second coupling element has a corresponding external thread section
and
is arranged or constructed on the shaped body, and wherein the at least one second coupling element is arranged or constructed on the grip element.

16. Medical instrumentation in accordance with claim 11, wherein the grip element has an elongate shaft, and wherein a gripping area is arranged or formed at a proximal end area of the shaft.

17. Medical instrumentation in accordance with claim 16, wherein the shaft defines a longitudinal axis, and wherein a proximal end face of the shaft is constructed in the form of an impact surface for an impactor tool.

18. Medical instrumentation in accordance with claim 17, wherein the impact surface is at least partially flat and defines a plane extending perpendicularly to the longitudinal axis.

19. Medical instrumentation in accordance with claim 16, wherein the shaft is of at least two-part construction and comprises a shaft sleeve and an inner shaft guided in the shaft sleeve, and wherein the second coupling element is arranged or formed at a distal end of the inner shaft or at a distal end of the shaft sleeve.

20. Medical instrumentation in accordance with claim 1, characterized by an impactor tool.
